(19) United States
(12) Patent Application Publication
(10) Pub. No.: US 2012/0071700 A1
(43) Pub. Date: Mar. 22, 2012

(54) CATALYSTS FOR HYDROGENATION OF UNSATURATED HYDROCARBONS AND PREPARATIONS AND USES THEREOF

(76) Inventors: Long Huang, Beijing (CN); Wei Dai, Beijing (CN); Baoliang Tian, Beijing (CN); Hui Peng, Beijing (CN); Guoqi Tang, Beijing (CN)

(21) Appl. No.: 13/238,765

(22) Filed: Sep. 21, 2011

(30) Foreign Application Priority Data
Sep. 21, 2010 (CN) 201010291704.0

(51) Int. Cl. C07C 7/167; B01J 37/08; B01J 31/38; B01J 37/16; B01J 31/28; B01J 27/13

(52) U.S. Cl. 585/259; 502/158

(57) ABSTRACT

Disclosed are catalysts for hydrogenation of unsaturated hydrocarbons comprising at least one carrier, at least one active metallic component supported on the at least one carrier, and at least one silane group; and wherein the at least one active metallic component is chosen from palladium, platinum, nickel, copper, and ruthenium; the at least one silane group is grafted to the catalyst by silylation and is present in an amount ranging from 0.05% to 25% by weight relative to the total weight of the catalyst. Also disclosed are processes for the preparation of the catalysts and processes using the catalysts for hydrogenation of unsaturated hydrocarbons.

This application claims priority under 35 U.S.C. §119 to Chinese Patent Application No. 201010291704.0, filed Sep. 21, 2010.

The present disclosure relates to catalysts for hydrogenation of unsaturated hydrocarbons and the preparations and uses thereof.

Hydrogenation of unsaturated hydrocarbons is an important reaction in the chemical industry. For example, hydrogenated saturation of olefin, selective hydrogenation of alkyne and diolefin into monoolefin, hydrogenation or selective hydrogenation of benzene ring and so on all have large-scale commercial applications (Jens Hagen, Industrial Catalysis: A Practical Approach(II), pp. 285-288 (2006)). The hydrogenation catalysts presently used in industry are mainly the support-type metal catalysts, and the active components include, for example, a metallic single-phase such as palladium, nickel, copper, and cobalt, or a metal sulfide. To improve the activity or selectivity of catalyst, a certain amount of metallic promoters are often added.

For these metal catalysts, the presence of water can reduce the hydrogenation activity of the catalyst considerably. And water may even reduce the working life of the catalyst. For example, Meille et al. inspected the effect of water on the Pd/Al2O3-catalyzed styrene hydrogenation reaction and showed that 100 ppm water in feed would be enough to reduce the catalytic activity to ⅓ of the original one (Valerie Meille and Claude de Bellefon, the Canadian Journal of Chemical Engineering, Volume 82, pp. 190-193 (2004)).

In the hydrogenation process of unsaturated hydrocarbons, the presence of water is often unavoidable in reaction processes such as hydrogenation of cracked gasoline, hydrogenation of C5-fractions in steam cracking, and hydrogenation of benzene. As a result, many catalysts have reduced reactivity and life in industrial operation. In addition, water content in a reactor may often change randomly in commercial operation, and sudden changes in water content could affect the hydrogenation capability of the catalyst and cause instability of the reaction process. This undoubtedly can increase the difficulties for operators and also reduce the safety of the process.

It is known that the increase of the catalyst life is critical to the enhancement of efficiency, energy consumption, and economic benefits of reaction devices. The published literatures show, for instance, that polymers produced from polymerization of olefins, such as diolefins, may cover the hydrogenation active sites of the catalyst and reduce the activity of the catalyst; meanwhile, pore channels would be blocked, which would reduce the diffusion coefficient of the catalyst and also the reactive performance of the catalyst (F. Schuth, J. Weitkamp, Handbook of Heterogeneous Catalysis: Second Edition, pp. 3266-3308 (2008)). Therefore, deposited carbon is often an important or a major cause for the deactivation of the catalyst used for hydrogenation of unsaturated hydrocarbons. For a highly unsaturated hydrocarbon selective hydrogenation catalyst, the presence of deposited carbon may further reduce selectivity. For example, in the C2 fronted end hydrogenation in ethylene plants, the deposited carbon produced from reaction not only reduces the hydrogenation activity of the catalyst, but also reduces the olefin selectivity in the selective hydrogenation reaction of alkynes and diolefins (M. Larsson, J. Jansson, S. Asplund, J, Catal., 178(1), pp. 49-57 (1998)).

It is known that a catalyst, such as a support-type catalyst, usually has catalyst dust from surface layer, which is unfavorable for the user of the catalyst. For instance, during the process of filling catalyst, catalyst dust comes from the metal catalyst would be a great threat to the health of operators; during the reaction process, such as the reaction in the presence of liquid phase, the dust of catalyst may be washed by solvent and later get into the downstream pipelines, causing the blocking of downstream pipelines or the like; meanwhile, severe catalyst dust could also cause the pressure of the catalyst bed layer to increase. Furthermore, after the regeneration of the catalyst, dust could even cause the reactor to stop from normal operation to replace the catalyst. Consequently, reduction of catalyst dust is of significant practical meaning to users.

Various hydrogenation catalysts or methods for improving the properties of the catalysts have been disclosed (see e.g., Chinese Patent No. 101429453, U.S. Pat. No. 6,013,847, and Chinese Patent No. 1317364). Certain improvement methods do not start from addressing the issues relating to the adsorption of water and deposited carbon and improving the water resistance and anti-deposited carbon property of the catalyst. For example, some methods improve the anti-deposited carbon capability of the catalyst by a method such as adding a metallic promoter or changing the crystalline phase of the carrier, but the enhancement of the anti-deposited carbon capability of the catalyst is extremely limited. Moreover, methods directed to suppressing the generation of deposited carbon have led to catalysts with reducing activity or selectivity.

To inhibit the water adsorption, an effective method at present is the addition of other components such as organic chlorine, but these components, when exist as impurities, would cause certain difficulties to the subsequent separation. In the meantime, the prior art is mainly based on maintaining water content at a certain level, yet when the incorporation of a trace amount of water in pulse causes the changes to the activity or selectivity of the catalyst, the stability of the catalyst can hardly be maintained.

Accordingly, a constant need exists for developing a catalyst with improved water resistance and/or anti-deposited carbon property for hydrogenation of unsaturated hydrocarbons.

After extensive research, the present inventors have found that grafting at least one silane group onto the catalyst supported on a hydroxy-containing carrier may change the amount of water adsorbed on the catalyst and the strength of such adsorption. The present inventors have found that it is the number of surface hydroxyl groups of the catalyst that is closely related to the deposited carbon. Without being bound by any theories, the present inventors have noticed that the active hydrogen on the surface hydroxyl groups of the catalyst may have a promotion effect on the unsaturated bond polymerization in hydrocarbons, and the number of active hydrogen on the catalyst surface is largely decreased after silylation.

Thus, provided herein are catalysts for hydrogenation of unsaturated hydrocarbons in industry with improved water resistance and/or anti-deposited carbon properties. Also provided herein are the methods for preparing such catalysts and their uses in hydrogenation of unsaturated hydrocarbons.

Disclosed herein is a catalyst for hydrogenation of unsaturated hydrocarbons comprising at least one carrier, at least one active metallic component supported on the at least one carrier, and at least one silane group, wherein the at least one silane group is grafted to the catalyst by silylation.

In comparison with the existing catalysts, the catalyst disclose herein can have advantages such as improved water resistance and anti-deposited carbon amount. For example, under the circumstance of containing water or where water appears possibly in pulse, the reactivity of this catalyst can be stable.

Further disclosed herein is a method for preparing a catalyst for hydrogenation of unsaturated hydrocarbons, comprising:
(1) supporting at least one active metallic component on at least one carrier to obtain a catalyst precursor I;
(2) reducing the at least one active metallic component to a metallic state or vulcanizing it to a vulcanized state to obtain a catalyst precursor II;
(3) silanizing the catalyst precursor II to graft the silane groups.

Further disclosed herein is the use of the catalyst for hydrogenation of unsaturated hydrocarbons. The hydrogenation catalyst can be used in selective hydrogenation and hydrogenated saturation with hydrocarbons as the main raw material.

### BRIEF DESCRIPTION OF DRAWINGS

FIG. 1 shows the C1s XPS patterns of Ni—Mg/Al₂O₃.

FIG. 2 shows the C1s XPS patterns of the catalyst Cat-1 of Example 1.

FIG. 3 shows the C1s XPS patterns of Pd—Ca/Al₂O₃—SiO₂.

FIG. 4 shows the C1s XPS patterns of the catalyst Cat-2 of Example 2.

Disclosed herein are catalysts for hydrogenation of unsaturated hydrocarbons, comprising at least one carrier, at least one active metallic component supported on the at least one carrier, and at least one silane group, wherein the at least one silane group is grafted to the at least carrier by silylation.

In some embodiments, the at least one active metallic component is chosen from palladium, platinum, nickel, copper, and ruthenium, and present in an amount ranging from 0.01% to 50% by weight relative to the total weight of the catalyst. For example, in one embodiment, the at least one metallic component is chosen from palladium, nickel, and copper, and present in an amount ranging from 0.05% to 45% by weight relative to the total weight of the catalyst.

In some embodiments, the main state of the at least one active metallic component under reaction conditions is in zero valent state. In some other embodiments, the at least one active metallic component is in the form of a metal sulfide.

In some embodiments, the catalyst may further comprise at least one metallic promoter (a), wherein the at least one metallic promoter (a) comprises more than one metallic element chosen from Groups IA, IIA, IIIA, IVA, and VA, and present in an amount ranging from 0.01% to 10% by weight relative to the total weight of the catalyst. For example, in one embodiment, the at least one metallic promoter (a) comprises more than one metallic element chosen from sodium, potassium, cesium, calcium, magnesium, barium, gallium, indium, lead, and bismuth, and present in an amount ranging from 0.01% to 6% by weight relative to the total weight of the catalyst.

In some embodiments, the catalyst may further comprise at least one metallic promoter (b), wherein the at least one metallic promoter (b) comprises more than one metallic element chosen from Groups IB, IIB, IIIB, and VIB, and present in an amount ranging from 0.01% to 10% by weight relative to the total weight of the catalyst. For instance, in one embodiment, the at least one metallic promoter (b) comprises more than one metallic element chosen from copper, silver, gold, zinc, mercury, lanthanum, thorium, cerium, chromium, molybdenum, and tungsten, and present in an amount ranging from 0.05% to 6% by weight relative to the total weight of the catalyst.

In some embodiments, the catalyst may further comprise at least one non-metallic promoter (c), wherein the at least one non-metallic promoter (c) comprises more than one non-metallic element chosen from Groups IIIA, IVA, and VA, and present in an amount ranging from 0.01% to 8% by weight relative to the total weight of the catalyst. For example, in one embodiment, the at least one non-metallic promoter (c) comprises more than one non-metallic element chosen from boron, phosphorous, sulphur, selenium, fluorine, chlorine, and iodine, and present in an amount ranging from 0.01% to 4% by weight relative to the total weight of the catalyst.

In general, any carrier known in the art can be used for the catalyst disclosed herein. In some embodiments, the at least one carrier is chosen from Al₂O₃, TiO₂, V₂O₅, SiO₂, ZnO, SnO₂, ZrO₂, MgO, activated carbon, kaolin, diatomite, and the mixtures of more than two of them. In some embodiments, the at least one carrier is a composite carrier formed from supporting on an inert substrate at least one of Al₂O₃, TiO₂, V₂O₅, SiO₂, ZnO, SnO₂, and MgO, wherein the inert substrate is chosen, for example, from metallic substrates and ceramic. In further some embodiments, the at least one carrier is chosen from Al₂O₃, TiO₂, ZrO₂, ZnO, MgO, activated carbon, diatomite, and the mixtures of more than two of them. The mixtures described herein include but not limited to: mechanical mixtures and/or the mixed oxides with chemical bond, such as Al₂O₃—SiO₂.

The texture performance, such as the pore size distribution, of the catalyst carrier may have influence on the performance of the catalyst. In some embodiments, the at least one carrier disclosed herein has a specific surface area ranging from 2 m²/g to 300 m²/g, such as from 5 m²/g to 180 m2/g, and a pore volume ranging from 0.05 ml/g to 1.2 ml/g, such as from 0.1 ml/g to 0.8 ml/g. In some embodiments, the pore size distribution of the carrier is as follows: the average pore size is more than 9 nm, and the pores with a pore size of more than 9 nm make up more than 50% of the pore volume and the pores with a pore size of less than 5 nm make up less than 25% of the pore volume. For example, in one embodiment, the average pore size is more than 11 nm, and the pores with a pore size of more than 11 nm make up more than 50% of the pore volume, and the pores with a pore size of less than 5 nm make up less than 10% of the pore volume.

The specific surface area, pore volume, and pore size distribution can be measured by the methods known to a person skilled in the art; for example, mercury injection apparatus, such as the automatic mercury injection apparatus manufactured by Quantachrome Company of U.S. (Type AUTOPORE IV 9510) can be used for measurement.

The at least one silane group in the catalyst is grafted to the catalyst by silylation. In some embodiments, the at least one silane group is grafted by silylation with at least one silanizing agent. The at least one silane group is present in an amount ranging from 0.05% to 25% by weight, such as from 0.1% to 15% by weight, relative to the total weight of the catalyst.

In some embodiments, in the silylation process, the at least one silanizing agent is chosen from organic silanes, organic siloxanes, organic silazanes, and organic chlorosilanes and the mixtures of more than two of them, for example, chosen from organic siloxanes, organic silazanes, and the mixtures thereof.

The catalyst for hydrogenation of unsaturated hydrocarbons of the various embodiments of the present disclosure can be used in the catalytic hydrogenation reaction of unsaturated hydrocarbons, for instance, in the catalytic hydrogenation reaction in which the hydrocarbons are present in an amount ranging from 50% to 100% by weight relative to the total weight of the feedstock.

In some embodiments, the catalyst for hydrogenation of unsaturated hydrocarbons of the present disclosure can be used in the following hydrogenation reactions: selective hydrogenation of alkynes and/or diolefins in the C2-fractions, C3-fractions, and/or C4-fractions generated in steam cracking, catalytic cracking, or thermal cracking process; selective hydrogenation of the stream rich in butadiene and pentadiene to remove alkynes; selective hydrogenation of gasoline to remove diolefins; hydrogenation of gasoline to remove olefins; hydrogenation and selective hydrogenation of benzene; hydrogenation of C4-raffinate, C5-raffinate, C9-raffinate, and arene raffinate; and hydrogenation of reformed oil.

In some embodiments, the compositions of the hydrogenation catalyst disclosed herein comprises, for example, in addition to the at least one silane group, Pd/Al₂O₃, Pd—Ag/Al₂O₃, Pd—Ag—K/Al₂O₃, Pd/MgAl₂O₄, Pd—Ag/SiO₂, Pd/activated carbon, Cu/SiO₂, Cu/ZnO—Al₂O₃, Ni—Ca/Al₂O₃, Pd—Ca/Al₂O₃, Ni/Al₂O₃, Ni—Co/Al₂O₃, Ni/diatomite, Ni—Mo—S/Al₂O₃, Ni/ZrO₂—TiO₂, Pt—K/Al₂O₃, Ru—Sn/Al₂O₃, Ru/activated carbon, and/or Ru/SiO₂.

Although the grafting of silane groups on the catalyst surface is not completely clear, it is possible to predict the form of the silane group based on the molecular structure of the silanizing agent and the principle of the silylation reaction. Below are the examples of the existing forms of some silane groups being grafted onto the catalyst.

The silane groups may be of the following general formula (1):

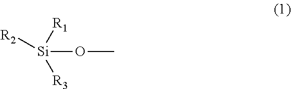

wherein, the substituents R₁, R₂ and R₃, which may be each independently the same or different, are chosen from alkyl groups, for example, methyl, ethyl, propyl, isopropyl, butyl, isobutyl, and cyclohexyl groups. Meanwhile, depending on whether there is a requirement for the selectivity of the reaction, the alkyl group may also be aromatic; another covalent bond of oxygen atom linked to Si is linked to the catalyst, and the silane groups are grafted onto the catalyst via the covalent bond of said oxygen atom.

The silane groups may also be of the following general formula (2):

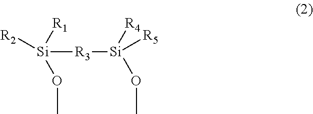

wherein, the substituents R₁, R₂, R₄ and R₅, which may be each independently the same or different, are chosen from alkyl groups, for example, methyl, ethyl, propyl, isopropyl, butyl, isobutyl, and cyclohexyl groups. Meanwhile, depending on whether there is a requirement for the selectivity of reaction, the alkyl group may also be aromatic; substituent R₃ may be chosen from chlorine, nitrogen, and oxygen. Another covalent bond of oxygen atom linked to Si is linked to the catalyst, and the silane groups are grafted onto the catalyst via the covalent bond of said oxygen atom.

The silane groups may also be of the following general formula (3):

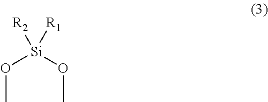

wherein, the substituents R₁ and R₂, which may be each independently the same or different, are chosen from alkyl groups, for example, methyl, ethyl, propyl, isopropyl, butyl, isobutyl, and cyclohexyl groups. Meanwhile, depending on whether there is a requirement for the selectivity of reaction, the alkyl group may also be aromatic; another covalent bond of oxygen atom linked to Si is linked to the catalyst, and the silane groups are grafted onto the catalyst via the covalent bond of said oxygen atom.

There may be a number of manners of supporting/adding a metallic component onto a carrier. For example, in one embodiment, impregnating the carrier with a solution or a suspension of the salt or oxide of a metal element and then drying. After drying, heating to a temperature ranging from 300° C. to 600° C. for calcination to obtain a metal oxide. The calcination atmosphere may be chosen from air, nitrogen, oxygen, argon, and a mixture thereof. In yet some embodiments, for metals that can be reduced to metallic state, after the metallic component is added to or supported on the catalyst, such as palladium, nickel, ruthenium, and copper, hydrogen or a hydrogen-containing gas may be used to reduce the catalyst and a liquid reducing agent chosen, for example, from methanol, isopropanol, formic acid, and hydrazine hydrate, may also be used to reduce the metal to a metallic state. For catalysts in which the metals such as cobalt and nickel may be present in the form of metal sulfides as the active component, the catalysts may be subjected to vulcanization by the existing precuring technique.

In another embodiment, the method of supporting/adding a metallic component comprises: impregnating the at least one carrier with a solution or a suspension of the salt or oxide of a metal element and then drying. After drying, at least one reducing agent may be further used to reduce the metallic component, wholly or in part, to a metallic state of zero valency. The reducing agent may be chosen, for example, from hydrogen, a hydrogen-containing gas, polyols, and hydrazine. For example, the at least one reducing agent is chosen from hydrogen-containing gas and polyols. The reducing agent may reduce the active metal compound to a corresponding metal or a compound of a lower valency. The catalyst precursor obtained by such a way may be subjected to another reduction with hydrogen before silylation, or may also be directly subjected to the silylation.

In some embodiments, the metallic component may also be supported on the carrier by the manners such as spraying, vaporization of metal or metal organics, and uniform deposition, and the active metallic component of the catalyst may be further transformed into the corresponding metallic state or vulcanized state by a manner of reduction or precuring. The methods for supporting/adding a metallic component onto the catalyst described above are provided as examples only, and in no way limiting the scope of the present disclosure.

In some embodiments, at least one promoter may be further supported/added on the carrier by the supporting method described above for the metallic component to further improve the hydrogenation property of the catalyst. The addition of the at least one promoter may be before or after supporting the active metal, or at the same time of adding the active metal. The addition of the at least one promoter may also be during the formation process of the carrier. During the formation process of the carrier, the salt or oxide of the metal promoter may be added and dispersed on the catalyst.

Since the silanizing agent has a relatively high reactivity, the specific reactions during the silylation process are not totally understood yet. According to the empirical principle obtained from the application of silylation reaction in chromatogram, during the silylation process, silane groups are grafted onto the catalyst surface by subjecting the silanizing agent and the hydroxy group on the catalyst surface to condensation reaction by silylation. The principle with organic siloxane as the silanizing agent is exemplified as follows:

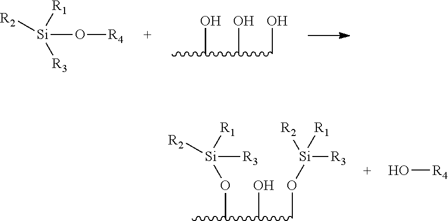

wherein the substituents R₁, R₂, R₃ and R₄ are defined as above.

In some embodiments, the grafting process may be carried out in a liquid solvent. The solvent may be chosen from ketones, ethers, hydrocarbons, and esters, for example, ethers and hydrocarbons. For instance, the solvent may be chosen from toluene, benzene, xylene, cyclohexane, hexane, heptane, ethyl ether, methyl phenoxide, tetrahydrofuran, liquid paraffin, saturated gasoline, hydrogenated saturated diesel, petroleum ether, and the mixtures thereof. In some embodiments, the grafting process may be carried out at a temperature ranging from 30° C. to 320° C., such as from 50° C. to 180° C.

In some other embodiments, the grafting of silane group may be carried out by a method comprising: in a carrier gas, bringing the silanizing agent in the form of gas or droplet into contact with the catalyst so as to complete the silylation of the catalyst. The carrier gas may be chosen, for example, from nitrogen, air, hydrogen, oxygen, carbon dioxide, argon, and the mixtures of more than two of them. In some catalyst-producing plants with restricted operation conditions, the silanizing agent may also be heated to become a steam and then be brought to be in contact with the catalyst for carrying out the grafting of silane group in the absence of carrier gas. When this method is used for grafting, the temperature is controlled at a temperature ranging from 60° C. to 450° C., such as from 85° C. to 280° C.

In some embodiments, the silanizing agent may be chosen from organic silanes, organic siloxanes, organic silazanes, and organic chlorosilanes, for example, methyltriethoxysilane, dimethyldiethoxysilane, trimethyldiethoxysilane, ethyltriethoxysilane, diethyldiethoxysilane, triethylethoxysilane, ethyltrimethoxysilane, butyltriethoxysilane, dimethylethylmethoxysilane, dimethylphenylethoxysilane, tripropylmethoxysilane, trimethylchlorosilane, dimethyldichlorosilane, dimethylpropylchlorosilane, dimethylbutylchlorosilane, dimethylisopropylchlorosilane, tributylchlorosilane, hexamethyldisilazane, heptamethyldisilazane, tetramethyldisilazane, 1,3-dimethyldiethyldisilazane, and 1,3-diphenyltetramethyldisilazane.

The coverage of the silane group on the catalyst surface may affect water resistance and anti-deposited carbon property of the catalyst disclosed herein. For example, when the coverage is low, the water resistance and the anti-deposited carbon property may not be put into full play; when the coverage is too high, polymerization may occur between silanes, which may cover the surface active sites of the catalyst and reduce the activity of the catalyst. Accordingly, in some embodiments, the content of the silane groups in the catalyst may be controlled to be present in an amount ranging from 0.05% to 25% by weight, such as from 0.1% to 15% by weight, relative to the total weight of the catalyst. The coverage of the silane groups may be controlled by the methods such as adjusting the amount of silanizing agent, silylation time, silylation temperature, the carrier-gas type, flow rate (gas-phase method), and solvent (liquid-phase method). When the gas-phase silylation is used, the residence time of the silanizing agent in the catalyst bed layer may be controlled to be a period of time ranging from 0.001 seconds to 400 seconds. The overall operation time of the gas-phase method may be a period of time ranging from 1 minute to 80 hours. Reduction in operation cost and operation time may be further achieved by adjusting the concentration of the silanizing agent. When the liquid-phase method is used, the residence time may be ranging from 0.5 seconds to 24 hours.

Also disclosed herein are methods for preparing the catalysts for hydrogenation of unsaturated hydrocarbons. In some embodiments, the method of preparing the catalysts is a gas-phase method using a carrier gas. The carrier gas in the silylation process may be chosen, for example, from nitrogen, air, hydrogen, oxygen, carbon dioxide, argon, methane, ethane, ethylene, propane, propylene, carbon monoxide, nitrogen oxides, and the mixtures thereof. In one embodiment, the carrier gas is chosen from nitrogen, hydrogen, argon, methane, and the mixtures thereof. The flow rate of the carrier gas may affect the residence time of the silanizing agent in the catalyst bed layer, and the result is calculated according to the ideal residence time model of reactor known in the art. In one embodiment, the residence time of the silanizing agent in the catalyst bed layer ranges from 0.0001 seconds to 400 seconds, such as from 0.001 seconds to 10.0 seconds. In some embodiments, the amount of the silanizing agent is not specifically defined, but may be present in an amount ranging from 0.01 g/L to 30 g/L.

As easily understood, the operations of supporting, reduction, and silylation of the active metal may be carried out in any catalyst-producing plants with suitable conditions.

In some embodiments, the methods of preparing the catalysts comprise on-line performing of reduction and silylation in a hydrogenation reactor. In one embodiment, the on-line method may comprise:
(1) reduction: keeping the reactor at a temperature ranging from 30° C. to 650° C., such as from 80° C. to 500° C.; introducing hydrogen or a mixed gas containing hydrogen to the catalyst; partially or completely reducing the active metallic component to a metallic state to achieve activation; and
(2) on-line silylation: keeping the reactor at a temperature ranging from 30° C. to 450° C., such as from 50° C. to 220° C.; grafting the silane groups onto the catalyst by contacting the silanizing agent, in the form of gas or droplet in a carrier gas, with the catalyst to carry out the on-line silylation; the contact time period may range from 15 minutes to 50 hours, such as from 0.5 hours to 20 hours, and the resulting grafted silane groups are present in an amount ranging from 0.05% to 25% by weight, such as from 0.5% to 15% by weight, relative to the total weight of the catalyst.

In some embodiments, the catalyst is exposed to or in contact with a gas stream containing water vapor for a period of time, such as from 0.5 hours to 30 hours, before silylation. In addition, in one embodiment, after the gas stream containing water vapor is no longer in contact with the catalyst, the reactor is kept at a temperature ranging from 50° C. to 200° C.; then a dry gas stream containing no water is introduced to dehydrate the catalyst for a period of time ranging, for example, from 0.5 hours to 40 hours to remove water physically adsorbed on the catalyst.

The sequence of the preparation operation of the catalyst may affect the activity and selectivity of the catalyst. Thus, in one embodiment, to avoid reducing the activity and selectivity of the catalyst, the operation of preparing the catalysts is conducted in the order described above.

The degree of coverage of the silane groups grafted onto the hydrogenation catalyst may be analyzed by X-ray photoelectron spectroscopy (XPS) to identify the carbon atom number on the catalyst surface and then calculate the surface coverage. Infrared spectroscopy (IR) may also be used to observe the functional groups on the catalyst surface, for example, the characteristic peak (˜2970 cm⁻¹) of —CH₃ is used to calculate the surface silane coverage degree; the characteristic peak (˜3750 cm⁻¹) of —OH is used to calculate the number of the residual hydroxyl groups on the catalyst surface. The organic carbon/elementary carbon (OC/EC) analyzer may be used to quantify the content of organic carbon so as to precisely obtain the amount of the silane groups on the catalyst.

In some embodiments, the catalysts disclosed herein unexpectedly has very little dust peeling phenomenon. In some cases, there is almost no generation of dust on the catalyst. In some other embodiments, the amount of the dust from the regeneration of the catalyst disclosed herein is relatively small in comparison with the catalyst in the prior art. Without being bound by the theory, the weak interaction between the supported metal and carrier is the cause of the generation of catalyst dust. When the catalyst is grafted with silane groups, the interaction between the pre-peeled dust and the carrier is strengthened by the chemical bonds between silane groups; thus, the amount of catalyst dust decreases greatly.

The catalyst disclosed herein can be used in the hydrogenation reaction of unsaturated hydrocarbons with hydrocarbons as the major raw material. The hydrogenation reaction may be double-bond olefins hydrogenation, alkynes or diolefins selective hydrogenation, or complete-hydrogenation of unsaturated hydrocarbons. The catalyst can be used in the reactions of the systems such as gas-liquid-solid, gas-solid phase, and gas-supercritical liquid phase-solid phase. As to the type of the reactor, the catalyst disclosed herein may be used on any of fixed bed, fluidized bed, slurry bed, moving bed, and magnetic suspension bed.

The hydrogenation catalyst disclosed herein may be used in the catalytic hydrogenation reaction of unsaturated hydrocarbons. For example, the catalyst disclosed herein may be used in: saturation of double bond in olefins into alkanes, selective hydrogenation of diolefins and alkynes into olefins, hydrogenation of diolefins and alkynes into alkanes, and hydrogenation of benzene ring into olefins or alkanes. In some embodiments, the catalyst disclosed herein is used for the catalytic hydrogenation reaction of the unsaturated hydrocarbons, and the raw materials of the hydrogenation reaction may comprise, for example, at least one of esters, ethers, alcohols, phenols, thiophenes, and furans, and hydrocarbons, but the major component in the raw materials is the hydrocarbon present in an amount ranging from 50% to 100% by weight relative to the total weight of the feedstock. In one embodiment, when the catalyst disclosed herein is used in the process of hydrogenation of unsaturated hydrocarbons, the upper limit of the water content allowable in the raw materials is 25% by weight relative to the total weight of the raw materials. Of course, in the case of higher water content, there already exists the remarkable delamination between water and unsaturated hydrocarbon. In the practical industrial operation, delamination separation is generally preferentially carried out.

In some embodiments, the hydrogenation reactions according to the present disclosure include: selective hydrogenation of alkynes and diolefins in the ethylene, propylene, or butylene stream in steam cracking, catalytic cracking, or thermal cracking process; selective hydrogenation of the alkynes in butadiene and pentadiene; selective hydrogenation of gasoline to remove diolefins; hydrogenation of gasoline to remove olefins; hydrogenation and selective hydrogenation of benzene; complete hydrogenation of cracked C4, C5, and C9 fractions; hydrogenation of cracked C4-raffinate, C5-raffinate, and C9-raffinate; hydrogenation of reformed oil.

The catalyst for hydrogenation of unsaturated hydrocarbons of the present disclosure may have one or more of the following advantages:
1. Increased water resistance. The reactive performance of the catalyst may not change drastically when a certain amount of water gets into raw materials by pulse or the water content in raw materials undergoes great changes. In some instances, the performance of the catalyst does not change markedly even when a small amount of alcohols or esters get into the reaction system.
2. The catalyst disclosed herein may greatly suppress the generation of polymers so as to reduce the amount of the deposited carbon generated during the reaction and largely improve the working life of the catalyst.
3. The dust of the catalyst disclosed herein may be reduced, which is favorable for the health of operators and also reduces the incidence of the problems such as blocking of system pipelines and increase of pressure drop of reactor.
4. The catalyst disclosed herein may partially utilize the existing catalyst-producing techniques and apparatuses; industrialization is simple, and the additional cost required for changing from the existing catalyst systems to the new system is relatively low.

### EXAMPLES

The present disclosure is further illustrated by the following examples, but is not limited by these examples.

### Example 1

50 g spherical Ni—Mg/Al₂O₃ catalyst with a diameter of 3 mm (manufactured by SINOPEC Beijing Research Institute of Chemical Industry; the volume is 72 ml; the weight percentages of Ni and Mg are 12% and 2.2%, respectively; the balance is Al₂O₃; the weight loss is 2.8 wt % when the temperature of the thermogravimetric analyzer rises to 500° C.; the specific surface area is 113 m²/g; the pore volume is 0.62 ml/g; the average pore size is 11.8 nm, wherein the pores with a pore size of more than 9 nm make up 69% of the pore volume and the pores with a pore size of less than 5 nm make up 10% of the pore volume) was fed into a fixed bed reactor (which had a diameter of 15 mm and a length of 400 mm and two temperature control points). First, the catalyst was reduced for 6 hours at 450° C. Then, the temperature of the reactor was decreased. While the temperature of the reactor was kept steady at 80° C., hydrogen gas containing 2% by volume of trimethylethoxysilane was introduced into the reactor at a flow rate of 300 ml/min. The temperature was maintained at 80° C. for 2 hours and was then raised to 120° C. After the temperature was stable at 120° C., it was maintained for 1 hour. Then, the introduction of the hydrogen gas containing trimethylethoxysilane was stopped. Nitrogen gas was then introduced to lower the temperature, and a catalyst Cat-1 was thus obtained.

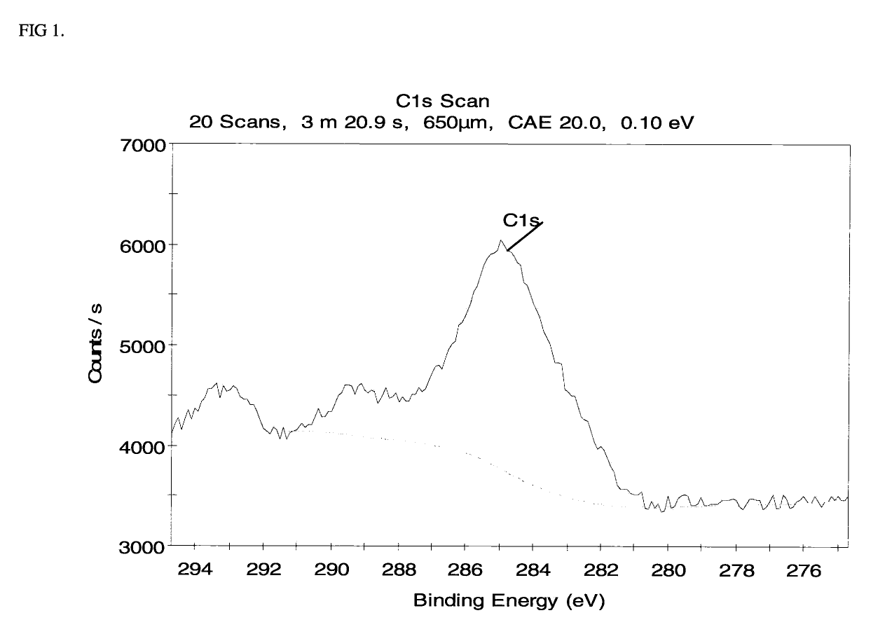

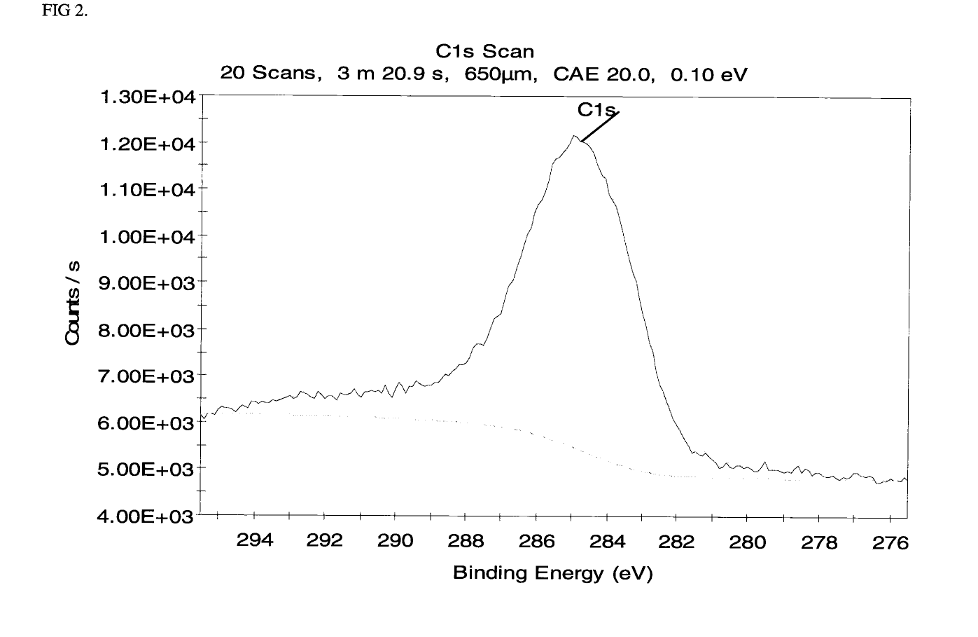

The untreated Ni—Mg/Al₂O₃ and Cat-1 were compared by a Fourier infrared spectroscopy (FTIR). The characteristic peak (˜2970 cm⁻¹) of methyl on Cat-1 was stronger than the untreated Ni—Mg/Al₂O₃, while the characteristic peak (˜3750 cm⁻¹) of hydroxy was greatly weaker than Ni—Mg/Al₂O₃. This showed that the hydroxy groups on Ni—Mg/Al₂O₃ were partially substituted by silane groups. Si content was quantitatively analyzed by an ICP-AES element analyzer, and was 1.8% by weight in Cat-1. Meanwhile, by the quantitative analysis with an organic carbon/elementary carbon (OC/EC) analyzer, the content of organic carbon was 2.31% by weight in Cat-1 and it can be calculated thereby that the weight percentage of the silane groups on the Cat-1 was 5.72% by weight. The catalyst surface analysis was carried out by using an X-ray photoelectron spectroscopy for the untreated Ni—Mg/Al₂O₃ and Cat-1. The surface C atom change was characterized to obtain the grafting situation of silane groups on the catalyst surface. The characterization patterns were respectively given in FIG. 1 and FIG. 2. It can also be clearly seen from FIG. 1 and FIG. 2 that carbon atoms of the catalyst surface increased after the silylation. This further demonstrated that the catalyst surface was grafted with silane groups.

### Comparative Example 1

50 g spherical Ni—Mg/Al₂O₃ catalyst with a diameter of 3 mm (manufactured by SINOPEC Beijing Research Institute of Chemical Industry, and as described in Example 1) was fed into a fixed bed reactor (which had a diameter of 15 mm and a length of 400 mm and two temperature exhibition control points). First, the catalyst was reduced for 6 h at 450° C. Then, the temperature of the reactor was lowered. While the temperature of the reactor was kept stable at 80° C., pure hydrogen gas (99.999%) was introduced into the reactor. The flow rate of the hydrogen gas was controlled at 300 ml/min. The temperature was maintained at 80° C. for 2 h and was later raised to 120° C. After the temperature was stable at 120° C., it was maintained for 1 h. Then, the introduction of the hydrogen was stopped. Nitrogen gas was then introduced to lower the temperature, and a catalyst Cat-2 was thus obtained.

The Ni—Mg/Al₂O₃ and Cat-2 were compared by a Fourier infrared spectroscopy (FTIR). Neither Cat-2 nor Ni—Mg/Al₂O₃ has the apparent characteristic peak (˜2970 cm⁻¹) of methyl, while the characteristic peak (˜3750 cm⁻¹) of hydroxy of Cat-2 was slightly weaker than that of Ni—Mg/Al₂O₃. Si content was quantitatively analyzed by an ICP-AES element analyzer, and was 0.005% by weight in Cat-2. Meanwhile, the content of organic carbon quantitatively analyzed by an organic carbon/elementary carbon (OC/EC) analyzer was at the lower limit of the instrument.

### Example 2

30 g strip-like Pd—Ca/Al₂O₃—SiO₂ catalyst with a diameter of 1.5 mm and a length of 1.5-5.0 mm (manufactured by SINOPEC Beijing Research Institute of Chemical Industry; the volume is 35 ml; the weight percentages of Pd and Ca are 0.08% and 0.5% respectively; the balance is Al₂O₃—SiO₂; the weight loss is 0.9 wt % when the temperature of the thermogravimetric analyzer rises to 300° C.; the specific surface area is 56 m²/g; the pore volume is 0.45 ml/g; the average pore size is 20.7 nm, wherein the pores with a pore size of more than 9 nm make up 83% of the pore volume and the pores with a pore size of less than 5 nm make up 2% of the pore volume) was fed into a 500 ml three-neck flask (one mouth was linked to a cooling coiled pipe, one mouth was linked to the temperature control exhibition point, and one mouth was a feeding mouth). 100 ml aqueous solution of isopropanol was added into the three-neck flask, and was subsequently put in an oil bath till the temperature increased to 180° C. and was maintained at 180° C. for 2 hours. Then, after filtering out the liquid, the catalyst was air dried. The three-neck flask was again put into a 110° C. oil bath, and 100 ml p-xylene containing 1.0 wt % trimethylchlorosilane was added into the flask. After the temperature was stable, it was maintained for 0.5 h. Then, after lowering the temperature of the three-neck flask, the catalyst was taken out, and dried in an oven at 160° C. for 3 h to obtain a catalyst Cat-3.

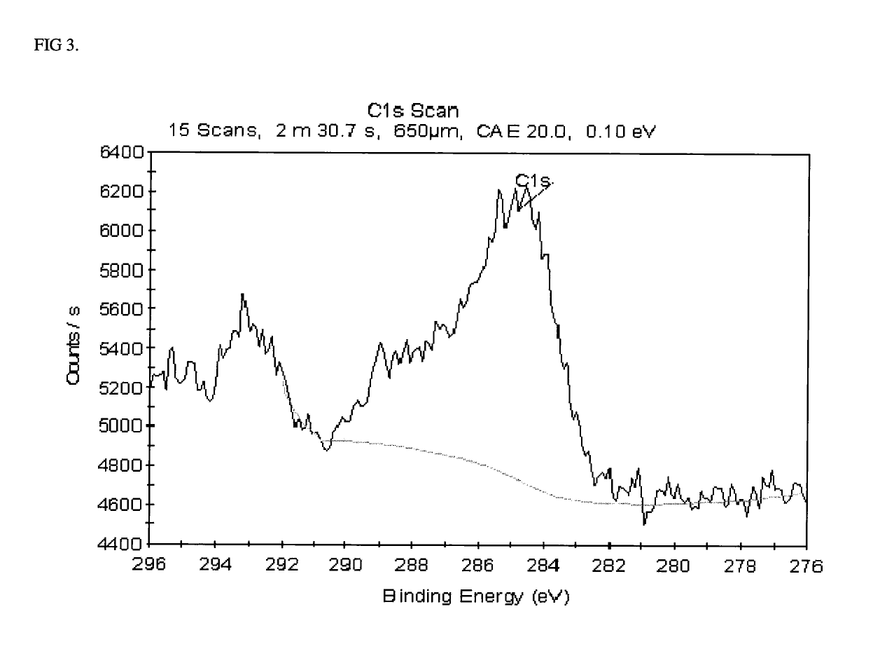

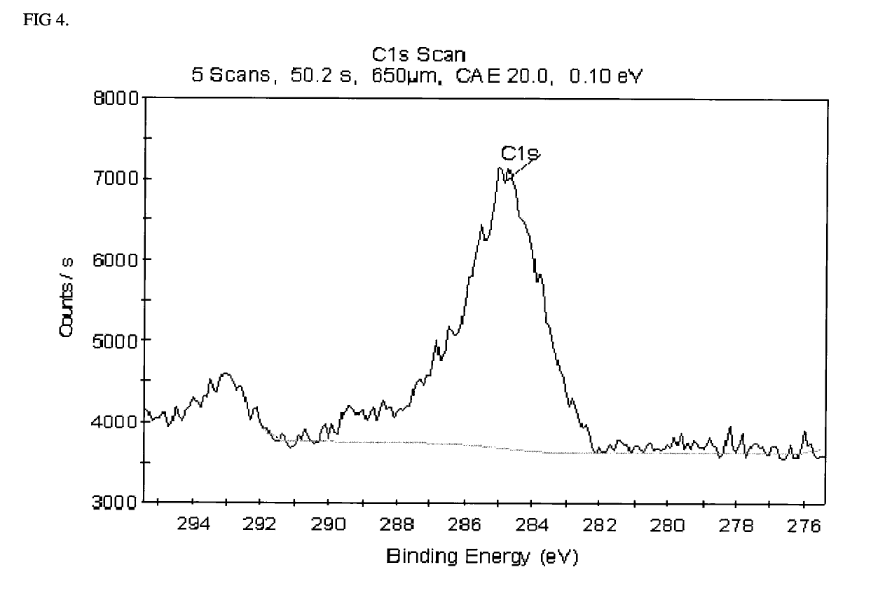

Pd—Ca/Al₂O₃—SiO₂ and Cat-3 were compared by a Fourier infrared spectroscopy (FTIR). The characteristic peak (2970 cm⁻¹) of methyl on Cat-3 was stronger than Pd—Ca/Al₂O₃—SiO₂, while the characteristic peak (˜3750 cm⁻¹) of hydroxy was evidently weaker than Pd—Ca/Al₂O₃—SiO₂. This showed that the hydroxy groups on Pd—Ca/Al₂O₃—SiO₂ were partially substituted by silane groups. The content of organic carbon quantitatively analyzed with an organic carbon/elementary carbon (OC/EC) analyzer was 1.0 wt % in Cat-3 and according to this, the weight percentage of the silane groups on Cat-3 was 2.25 wt %. The catalyst surface analysis was carried out by using an X-ray photoelectron spectroscopy for the untreated Pd—Ca/Al₂O₃—SiO₂ and Cat-3. The surface carbon atom change was characterized to obtain the grafting situation of silane groups on the catalyst surface. The characterization patterns were respectively given in FIG. 3 and FIG. 4. It can also be clearly seen from FIG. 3 and FIG. 4 that carbon atoms of the catalyst surface were increased after the silylation. This further demonstrated that the catalyst surface was grafted with silane groups.

### Comparative Example 2

30 g strip-like Pd—Ca/Al₂O₃—SiO₂ catalyst with a diameter of 1.5 mm and a length of 1.5-5.0 mm (as described in Example 2) was fed into a 500 ml three-neck flask (one mouth was linked to a cooling coiled pipe, one mouth was linked to the temperature control exhibition point, and one mouth was for feeding). The three-neck flask was put into a 110° C. oil bath, and 100 ml p-xylene (analytical reagent, with a concentration of >99.9%) was added into the flask. After the temperature was stable, it was maintained for 0.5 h. Then, after lowering the temperature of the three-neck flask, the catalyst was taken out, and later dried in an oven at 160° C. for 3 h to obtain the catalyst Cat-4.

The Pd—Ca/Al₂O₃—SiO₂ and Cat-4 were compared by a Fourier infrared spectroscopy (FTIR). Neither Cat-4 nor Pd—Ca/Al₂O₃—SiO₂ has the apparent characteristic peak (˜2970 cm⁻¹) of methyl, while the characteristic peak (˜3750 cm⁻¹) of hydroxy of Cat-4 was slightly weaker than that of Pd—Ca/Al₂O₃—SiO₂. Meanwhile, the content of organic carbon quantitatively analyzed by an organic carbon/elementary carbon (OC/EC) analyzer was at the lower limit of the instrument.

### Example 3

25 g strip-like Pd—Ni—La—Mg/ZrO₂—Al₂O₃ catalyst with a diameter of 3 mm (manufactured by SINOPEC Beijing Research Institute of Chemical Industry; the volume is 41 ml; the weight percentages of Pd, Ni, La and Mg are 0.02%, 5.0%, 0.8% and 2.8% respectively; the balance is ZrO₂—Al₂O₃; the weight loss is 1.8 wt % when the temperature of the thermogravimetric analyzer rises to 500° C.; the specific surface area is 34 m²/g; the pore volume is 0.51 ml/g; the average pore size is 60.8 nm, wherein the pores with a pore size of more than 9 nm make up 92% of the pore volume and the pores with a pore size of less than 5 nm make up 0.7% of the pore volume) was reduced for 3 h in a fixed bed at 140° C. in a hydrogen atmosphere. The treated catalyst was then added into a 500 ml three-neck flask, which was placed in an oil bath (one mouth was linked to a cooling coiled pipe, one mouth was linked to a thermometer, and one mouth was a feeding mouth). Then, 150 ml p-xylene was poured into the three-neck flask. After the temperature of the reactor was kept stable at 110° C., a hydrogen gas and 8 ml hexamethyldisilazane were introduced into the flask. The temperature was maintained at 110° C. for 1 h, and was then raised to 140° C. After the temperature was stable at 140° C., it was maintained for 1 h, and was then lowered. The catalyst was later taken out of the flask and dried in an oven at 160° C. for 3 h to obtain a catalyst Cat-5.

Pd—Ni—La—Mg/ZrO₂—Al₂O₃ and Cat-5 were compared by a Fourier infrared spectroscopy (FTIR). The characteristic peak (˜2970 cm⁻¹) of methyl on Cat-5 was stronger than Pd—Ni—La—Mg/ZrO₂—Al₂O₃, while the characteristic peak (˜3750 cm⁻¹) of hydroxy was weaker than Pd—Ni—La—Mg/ZrO₂—Al₂O₃. This showed that the hydroxy groups on Pd—Ni—La—Mg/ZrO₂—Al₂O₃ were partially substituted by silane groups. Si content was analyzed by an ICP-AES element analyzer, and was quantified as 0.8 wt % in Cat-5. Meanwhile, the content of organic carbon quantified by an organic carbon/elementary carbon (OC/EC) analyzer was 1.10 wt %, and according to this, the weight percentage of the silane groups on the catalyst was about 2.55 wt %.

### Comparative Example 3

25 g strip-like Pd—Ni—La—Mg/ZrO2-Al2O3 catalyst with a diameter of 3 mm (manufactured by SINOPEC Beijing Research Institute of Chemical Industry, as described in Example 3) was fed into a 500 ml three-neck flask, which was placed in an oil bath and of which one mouth was linked to a cooling coiled pipe, one mouth was linked to a thermometer, and one mouth was a feeding mouth. At first, 150 ml p-xylene was added into the three-neck flask, and the temperature of the reactor was kept stable at 110° C. for 1 h and was then raised to 140° C. After the temperature was stable at 140° C., it was maintained for 1 h and then lowered. The catalyst was taken out of the flask and then dried in an oven at 160° C. for 3 h to obtain a catalyst Cat-6.

### Example 4

40 g spherical Ru—Sn—K/Al₂O₃ catalyst with a diameter of 1.5 mm (manufactured by SINOPEC Beijing Research Institute of Chemical Industry; the volume is 52 ml; the weight percentages of Ru, Sn and K are 0.4%, 1.2% and 2.2% respectively; the balance is Al₂O₃; the weight loss is 1.9 wt % when the temperature of the thermogravimetric analyzer rises to 500° C.; the specific surface area is 160 m²/g; the pore volume is 0.77 ml/g; the average pore size is 10.8 nm, wherein the pores with a pore size of more than 9 nm make up 62% of the pore volume and the pores with a pore size of less than 5 nm make up 16% of the pore volume) was fed into a fixed bed reactor (which had a diameter of 15 mm and a length of 400 mm and two temperature exhibition control points). The catalyst was reduced in a mixed gas of nitrogen and hydrogen with 25 vol. % of hydrogen. The reduction temperature was 450° C., and the reduction time period was 5 h. Then the temperature was lowered by introducing nitrogen gas. After the temperature of the reactor was kept stable at 60° C., hydrogen gas containing 2 vol % dimethyldiethoxysilane and 1 vol % hexamethyldisilazane was introduced into the reactor at a flow rate of 200 ml/min. The temperature was maintained at 60° C. for 4 h and was then raised to 110° C. After the temperature was stable, it was maintained for 1 h. Then, the introduction of the hydrogen gas containing dimethyldiethoxysilane was stopped. Nitrogen gas was then introduced to lower the temperature, and a catalyst Cat-7 was thus obtained.

By analysis, the weight percentage of the silane groups on the catalyst was 2.81 wt %.

### Example 5

2.0 mm, spherical, alumina carrier (which contained 0.2 wt % and 1.5 wt % of La and K element promoters; the specific surface area of the carrier was 32 m²/g) was taken, and a catalyst precursor I (which contains Pd, Ag and Bi, each being 0.1 wt %; the pore volume was 0.40 ml/g; the average pore size was 72.3 nm, wherein the pores with a pore size of more than 9 nm made up 97% of the pore volume and the pores with a pore size of less than 5 nm made up 0.1% of the pore volume) was obtained by a manner of equivolume impregnation. 50 ml catalyst precursor I was fed into a fixed bed, and was then reduced in hydrogen for 3 h (all of the gas flows indicated below were kept constant at 150 ml/min). The reduction temperature was 180° C. Then the temperature was lowered to 120° C., and a hydrogen gas containing 1 vol % water vapour was introduced for the treatment of 2 h, and then was switched to the dry nitrogen for the purging of 3 h. Under the situation where a temperature of 120° C. was maintained, a hydrogen gas containing 2 vol % methyltriethoxysilane was introduced and was maintained for 0.5 h. After that, pure nitrogen was introduced for purging of 2 h at a temperature raised to 200° C. A catalyst Cat-8 was obtained upon lowering temperature.

By analysis, the weight percentage of the silane groups on the catalyst was 1.89 wt %.

### Example 6

30 g spherical Pd—Ag/Al₂O₃ catalyst with a diameter of 1.5 mm (manufactured by SINOPEC Beijing Research Institute of Chemical Industry; the volume is 35 ml; the weight percentages of Pd and Ag are 0.08% and 0.05% respectively; the balance is Al₂O₃; the specific surface area is 96 m²/g; the pore volume is 0.73 ml/g; the average pore size is 34.8 nm, wherein the pores with a pore size of more than 9 nm make up 77% of the pore volume and the pores with a pore size of less than 5 nm make up 0.8% of the pore volume) was fed into a 500 ml three-neck flask, of which one mouth was linked to a cooling coiled pipe, one mouth was linked to the temperature control exhibition point, and one mouth was a feeding mouth. The three-neck flask was placed in an oil bath at a temperature of 110° C., and 100 ml p-xylene containing 1.0 wt % hexamethyldisilazane was added. After the temperature was stable, it was maintained for 0.5 h. Then, the temperature of the three-mouth flask was lowered. The catalyst was taken out of the flask and dried in an oven at 160° C. for 3 h to obtain a catalyst Cat-9.

By analysis, the weight percentage of the silane groups on the catalyst was 2.61 wt %.

### Example 7

30 g spherical Cu—Pd—La—F/Al2O3 catalyst with a diameter of 1.5 mm (manufactured by SINOPEC Beijing Research Institute of Chemical Industry; the volume is 35 ml; the weight percentages of Cu, Pd, La and F are 5%, 0.06%, 0.1% and 0.08% respectively; the balance is Al2O3) was fed into a 500 ml three-neck flask, of which one mouth was linked to a cooling coiled pipe, one mouth was linked to the temperature control exhibition point, and one mouth was a feeding mouth. The three-mouth flask was placed in an oil bath at a temperature of 110° C., and 100 ml p-xylene containing 1.0 wt % tripropylmethoxysilane was poured into the flask. After the temperature was stable, it was maintained for 0.5 h. Then, the temperature of the three-neck flask was lowered. The catalyst was taken out of the flask and dried in an oven at 160° C. for 3 h to obtain a catalyst Cat-11.

By analysis, the weight percentage of the silane groups on the catalyst was 2.80 wt %.

### Example 8

2.0 mm, spherical alumina (the specific surface area is 221 m²/g; the pore volume is 0.88 ml/g; the average pore size is 16.8 nm, wherein the pores with a pore size of more than 9 nm make up 70% of the pore volume and the pores with a pore size of less than 5 nm make up 1.1% of the pore volume) was taken. 0.2 wt % aqueous solution of palladium chloride was supported on the catalyst by spraying, and then a catalyst precursor was obtained by calcination decomposition. 50 ml catalyst precursor was placed in a fixed bed for gas-phase selective hydrogenation of C3-fractions to remove propyne and propadiene, and was reduced for 3 h at 160° C. and with a 200 ml/min hydrogen, and was then cooled to 110° C. Subsequently, a nitrogen gas containing 5 vol % of trimethylethoxysilane was directly introduced for treatment of 3 h, and then the temperature was raised to 180° C. and nitrogen was introduced for purging of 5 h.

By analysis, the weight percentage of the silane groups on the catalyst was 9.90 wt %.

### Example 9

The catalyst of Example 1 and the catalyst of Comparative Example 1 were respectively used in the saturation reaction of the cracked C5-raffinate. The raw materials contained about 60 wt % of pentane; about 40 wt % of monolefin; and about 0.04 wt % of water. The hydrogenation reactor was an isothermal fixed bed. The process conditions of the hydrogenation reaction were: pressure: 1.0 MPa; inlet temperature: 190° C.; hydrogen/oil molar ratio: 4.5; liquid hourly space velocity: 2.0 h⁻¹. In the hydrogenation reaction, 5 ml water vapour was added into the reactor in pulse per 100 h to inspect the water resistance of the catalyst. After the completion of the reaction which lasted for 300 h, the deposited carbon amounts were compared by the combined use of thermal gravity-mass spectrum. The result was shown in Table 1. It showed that the catalyst of the present disclosure had a higher water resistance and a stronger anti-deposited carbon property in comparison with the comparative catalysts.

TABLE 1
Catalytic performance of Catalysts of Example 1 and Comparative
Example 1

| | | | | | | | Deposited |
|---|---|---|---|---|---|---|---|
| | | | | | | | carbon |
| | | Operation time (h) | | | | | amount |
| Catalyst | | 10 | 99 | 102 | 200 | 300 | (mg/g) |
| Example 1 | olefin | 99.5 | 99.6 | 99.3 | 99.4 | 99.3 | 31 |
| | conversion | | | | | | |
| | (%) | | | | | | |
| Comparative | olefin | 98.4 | 98.0 | 90.4 | 92.4 | 90.9 | 128 |
| Example 1 | conversion | | | | | | |
| | (%) | | | | | | |

Determination of deposited carbon amount: a thermal gravity-mass spectrometer; an air atmosphere of 30 ml/min; a temperature rising rate of 10° C./min; temperature rising from room temperature to 450° C.; the CO₂ peak in the mass spectrum was used to determine the position of the weight loss peak of the deposited carbon during thermal gravity test and the thermal gravity result was used to make quantification.

### Example 10

The catalyst of Example 2 and the catalyst of Comparative Example 2 were respectively used in the one-section selective hydrogenation reaction of the cracked gasoline. In the raw materials, diene value was 26.2×10⁻² g/g; the distillation range was 73-459° C.; the water content was 0.042 wt %. The hydrogenation reactor was an adiabatic fixed-bed integral reactor. The process conditions of the hydrogenation reaction were: pressure: 2.5 MPa; inlet temperature: 45° C.; hydrogen/oil molar ratio: 6.5; liquid hourly space velocity: 2.8 h⁻¹. After the completion of the reaction which lasted for 300 h, the deposited carbon amounts were compared by the combined use of thermal gravity-mass spectrum. The result was shown in Table 2. It showed that the catalyst of the present disclosure had a higher reactivity in the water-containing raw materials and a small amount of deposited carbon in comparison with the existing catalysts.

TABLE 2
Catalytic performance of Catalysts of Example 2 and Comparative
Example 2

| | | | | | | | Deposited |
|---|---|---|---|---|---|---|---|
| | | | | | | | carbon |
| | | Operation time (h) | | | | | amount |
| Catalyst | | 10 | 100 | 150 | 200 | 300 | (mg/g) |
| Example 2 | diolefin | 0.15 | 0.14 | 0.14 | 0.13 | 0.13 | 20 |
| | value | | | | | | |
| | (×10⁻² | | | | | | |
| | g/g) | | | | | | |
| Comparative | diolefin | 0.59 | 0.65 | 0.84 | 0.98 | 1.12 | 89 |
| Example 2 | value | | | | | | |
| | (×10⁻² | | | | | | |
| | g/g) | | | | | | |

Determination of deposited carbon amount: a thermal gravity-mass spectrometer; an air atmosphere of 30 ml/min; a temperature rising rate of 10° C./min; temperature rising from room temperature to 450° C.; the CO₂ peak in the mass spectrum was used to determine the position of the weight loss peak of the deposited carbon by thermal gravity, and the thermal gravity result was used to make quantification.

### Example 11

The catalyst of Example 3 and the catalyst of Comparative Example 3 were respectively applied to the selective hydrogenation reaction of acetylene. The raw materials contained 1.22 wt % of acetylene; and the molar ratio of hydrogen:acetylene was 1.07:1. The hydrogenation reactor was a 25 ml isothermal fixed bed. The catalyst was 3.0 g. The process conditions of the hydrogenation reaction were the same as those shown in Table 1. In the hydrogenation reaction, 2.0 ml water vapour was added in pulse after 150 h to inspect the water resistance of catalyst. After the completion of the reaction which lasted for 900 h, the deposited carbon amounts were compared by the combined use of thermal gravity-mass spectrum. Therein, the conversion and selectivity of acetylene were calculated by the following methods:

$${C}_{2} {H}_{2} Conversion = \frac{{( {C}_{2} {H}_{2} )}_{in} - {( {C}_{2} {H}_{2} )}_{out}}{{( {C}_{2} {H}_{2} )}_{in}} \times 100$$

$${C}_{2} {H}_{2} Selectivity = \frac{{( {C}_{2} {H}_{4} )}_{out} - {( {C}_{2} {H}_{4} )}_{in}}{{( {C}_{2} {H}_{2} )}_{in} - {( {C}_{2} {H}_{2} )}_{out}} \times 100$$

The results were as shown in Table 3. It showed that the method of the present disclosure, as compared to the existing methods, had a higher catalyst activity in the case where the raw materials contained water, and meanwhile, the higher adaptability in response to the sudden water content changes and the enhanced anti-deposited carbon ability of catalyst.

TABLE 3
Catalytic Performance of Catalysts of Example 3 and of Comparative
Example 3

| Catalyst | Example 3 | | | | | |
|---|---|---|---|---|---|---|
| Reaction | temperature: 60° C., reaction pressure: 1.0 MPa, | | | | | |
| conditions | gas space velocity: 5500 h⁻¹ | | | | | |
| | operation time | 50 | 148 | 155 | 500 | 900 |
| | (h) | | | | | |
| | conversion | 99.95 | 99.95 | 99.93 | 99.94 | 99.94 |
| | (mol %) | | | | | |
| | selectivity | 50.4 | 49.8 | 50.9 | 50.7 | 50.9 |
| | (mol %) | | | | | |
| | deposited carbon | 21 | | | | |
| | amount (mg/g) | | | | | |
| Catalyst | Comparative Example 3 | | | | | |
| Reaction | temperature: 65° C., reaction pressure: 1.3 MPa, | | | | | |
| conditions | gas space velocity: 8500 h⁻¹ | | | | | |
| | operation time | 50 | 148 | 155 | 500 | 900 |
| | (h) | | | | | |
| | conversion | 99.97 | 99.98 | 70.21 | 76.4 | 81.9 |
| | (mol %) | | | | | |
| | selectivity | 38.4 | 37.4 | 54.2 | 44.2 | 42.6 |
| | (mol %) | | | | | |

| | |
|---|---|
| deposited carbon | 121 |
| amount (mg/g) | |

Determination of deposited carbon amount: a thermal gravity-mass spectrometer; an air atmosphere of 30 ml/min; a temperature rising rate of 10° C./Min; temperature rising from room temperature to 450° C.; the CO₂ peak in the mass spectrum was used to determine the position of the weight loss peak of the deposited carbon by thermal gravity, and the thermal gravity result was used to make quantification.

What is claimed is:

1. A catalyst for hydrogenation of unsaturated hydrocarbons comprising at least one carrier, at least one active metallic component supported on the at least one carrier, and at least one silane group; wherein the at least one active metallic component is chosen from palladium, platinum, nickel, copper, and ruthenium; the at least one silane group is grafted to the catalyst by silylation and is present in an amount ranging from 0.05% to 25% by weight relative to the total weight of the catalyst.

2. The catalyst for hydrogenation of unsaturated hydrocarbons according to claim 1, wherein the at least one silane group is grafted to the catalyst by silylation and is present in an amount ranging from 0.1% to 15% by weight relative to the total weight of the catalyst.

3. The catalyst for hydrogenation of unsaturated hydrocarbons according to claim 1, wherein the at least one active metallic component is present in an amount ranging from 0.01% to 50% by weight relative to the total weight of the catalyst.

4. The catalyst for hydrogenation of unsaturated hydrocarbons according to claim 1, wherein the at least one active metallic component is chosen from palladium, nickel, and copper, and is present in an amount ranging from 0.05% to 45% by weight relative to the total weight of the catalyst.

5. The catalyst for hydrogenation of unsaturated hydrocarbons according to claim 1 further comprises at least one metallic promoter (a) comprising more than one metallic element chosen from Groups IA, IIA, IIIA, IVA, and VA, and is present in an amount ranging from 0.01% to 10% by weight relative to the total weight of the catalyst.

6. The catalyst for hydrogenation of unsaturated hydrocarbons according to claim 5, wherein the at least one metallic promoter (a) comprises more than one metallic element chosen from sodium, potassium, cesium, calcium, magnesium, barium, gallium, indium, lead, and bismuth, and is present in an amount ranging from 0.01% to 6% by weight relative to the total weight of the catalyst.

7. The catalyst for hydrogenation of unsaturated hydrocarbons according to claim 1 further comprises at least one metallic promoter (b) comprising more than one metallic element chosen from Groups IB, IIB, IIIB, and VIB, and is present in an amount ranging from 0.01% to 10% by weight relative to the total weight of the catalyst.

8. The catalyst for hydrogenation of unsaturated hydrocarbons according to claim 7, wherein the at least one metallic promoter (b) comprises more than one metallic element chosen from copper, silver, gold, zinc, mercury, lanthanum, thorium, cerium, chromium, molybdenum, and tungsten, and is present in an amount ranging from 0.05% to 6% by weight relative to the total weight of the catalyst.

9. The catalyst for hydrogenation of unsaturated hydrocarbons according to claim 1 further comprises at least one non-metallic promoter (c) comprising more than one non-metallic element chosen from Groups IIIA, IVA, and VA, and is present in an amount ranging from 0.01% to 8% by weight relative to the total weight of the catalyst.

10. The catalyst for hydrogenation of unsaturated hydrocarbons according to claim 9, wherein the at least one non-metallic promoter (c) comprises more than one non-metallic element chosen from boron, phosphorous, sulphur, selenium, fluorine, chlorine, and iodine, and is present in an amount ranging from 0.01% to 4% by weight relative to the total weight of the catalyst.

11. The catalyst for hydrogenation of unsaturated hydrocarbons according to claim 1, wherein the at least one carrier is chosen from Al₂O₃, TiO₂, V₂O₅, SiO₂, ZnO, SnO₂, ZrO₂, MgO, activated carbon, kaolin, and diatomite.

12. The catalyst for hydrogenation of unsaturated hydrocarbons according to claim 1, wherein the at least one carrier is chosen from composite carriers formed from supporting on an inert substrate at least one of Al₂O₃, TiO₂, V₂O₅, SiO₂, ZnO, SnO₂, and MgO; the inert substrate is chosen from metallic substrates and ceramic.

13. The catalyst for hydrogenation of unsaturated hydrocarbons according to claim 11, wherein the at least one carrier is chosen from Al₂O₃, TiO₂, ZrO₂, ZnO, MgO, activated carbon, and diatomite.

14. The catalyst for hydrogenation of unsaturated hydrocarbons according to claim 1, wherein the at least one carrier has a specific surface area ranging from 2 m²/g to 300 m²/g, a pore volume ranging from 0.05 ml/g to 1.2 ml/g, and an average pore size of more than 9 nm, and the pores with a pore size of more than 9 nm are present in an amount of more than 50% of the pore volume and the pores with a pore size of less than 5 nm are present in an amount of less than 25% of the pore volume.

15. A process for preparing a catalyst for hydrogenation of unsaturated hydrocarbons according to claim 1, comprising:
(1) supporting at least one active metal component on at least one carrier to obtain a catalyst precursor I;
(2) reducing said at least one active metal component to a metallic state or vulcanizing it to a vulcanized state to obtain a catalyst precursor II; and
(3) grafting at least one silane group to the catalyst precursor II.

16. The process according to claim 15, wherein the silylation is performed in a gas phase by contacting at least one silanizing agent with the catalyst precursor II by a carrier gas or by gasifying the silanizing agent itself.

17. The process according to claim 15, wherein the catalyst precursor II is in contact with a gas stream containing water vapor for a period of time ranging from 0.5 h to 30 h before silylation.

18. The process according to claim 15, wherein the operations (2) and (3) are carried out on line after the catalyst precursor I is fed into a reactor.

19. The process according to claim 15, wherein the operation (3) is carried out on line after the catalyst precursor II is fed into a reactor.

20. The process according to claim 15, wherein:
the reduction operation (2) comprises keeping a reactor at a temperature ranging from 30° C. to 650° C.; contacting hydrogen gas or a mixed gas containing hydrogen with the catalyst precursor I; and wholly or partially reducing the at least one active metallic component to a metallic state to achieve activation;
the silylation operation (3) comprises keeping the reactor at a temperature ranging from 30° C. to 450° C.; contacting at least one silanizing agent in a form of gas or droplet in a carrier gas with the catalyst precursor II for a period of time ranging from 15 minutes to 50 h; and
further wherein the at least one silane group is present in an amount ranging from 0.05% to 25% by weight relative to the total weight of the catalyst.

21. The process according to claim 16 or 20, wherein: the carrier gas for the silylation is chosen from nitrogen, air, hydrogen, oxygen, carbon dioxide, argon, methane, ethane, ethylene, propane, propylene, carbon monoxide, nitrogen oxides, and the mixtures thereof.

22. The process according to claim 21, wherein the carrier gas for the silylation is chosen from nitrogen, hydrogen, argon, methane, and the mixtures thereof.

23. The process according to claim 16 or 20, wherein the at least one silanizing agent is chosen from organic silanes, organic siloxanes, organic silazanes, and organic chlorosilanes.

24. The process according to claim 23, wherein the at least one silanizing agent is chosen from methyltriethoxysilane, dimethyldiethoxysilane, trimethyldiethoxysilane, ethyltriethoxysilane, diethyldiethoxysilane, triethylethoxysilane, ethyltrimethoxysilane, butyltriethoxysilane, dimethylethylmethoxysilane, dimethylphenylethoxysilane, tripropylmethoxysilane, trimethylchlorosilane, dimethyldichlorosilane, dimethylpropylchlorosilane, dimethylbutylchlorosilane, dimethylisopropylchlorosilane, tributylchlorosilane, hexamethyldisilazane, heptamethyldisilazane, tetramethyldisilazane, 1,3-dimethyldiethyldisilazane, and 1,3-diphenyltetramethyldisilazane.

25. A process for hydrogenation of unsaturated hydrocarbons comprising using a catalyst in a catalytic hydrogenation reaction of unsaturated hydrocarbons, wherein the catalyst comprising at least one carrier, at least one active metallic component supported on the at least one carrier, and at least one silane group; and wherein the at least one active metallic component is chosen from palladium, platinum, nickel, copper, and ruthenium; the at least one silane group is grafted to the catalyst by silylation and is present in an amount ranging from 0.05% to 25% by weight relative to the total weight of the catalyst.

26. The process for hydrogenation of unsaturated hydrocarbons according to claim 25 further comprises using a feedstock comprising hydrocarbons present in an amount ranging from 50% to 100% by weight relative to the total weight of the feedstock.

27. The process for hydrogenation of unsaturated hydrocarbons according to claim 25, wherein the catalytic hydrogenation reaction is chosen from selective hydrogenation of alkynes and/or diolefins in the C2-fractions, C3-fractions, and/or C4-fractions generated in steam cracking, catalytic cracking, or thermal cracking process; selective hydrogenation of the stream rich in butadiene and pentadiene to remove alkynes; selective hydrogenation of gasoline to remove diolefins; hydrogenation of gasoline to reduce olefins; hydrogenation and selective hydrogenation of benzene; hydrogenation of C4-raffinate, C5-raffinate, C9-raffinate, and arene raffinate; and hydrogenation of reformed oil.
